(19) United States
(12) Patent Application Publication
(10) Pub. No.: US 2016/0083340 A1
(43) Pub. Date: Mar. 24, 2016

(54) METHOD OF PURIFYING DIMETHYL SULFOXIDE

(71) Applicant: TORAY FINE CHEMICALS CO., LTD., Chiyoda-ku, Tokyo (JP)

(72) Inventors: Michiko Ishino, Tokai (JP); Koji Tanaka, Moriyama (JP); Hiromitsu Abe, Tokai (JP); Hideki Inoue, Moriyama (JP)

(21) Appl. No.: 14/786,235

(22) PCT Filed: Apr. 22, 2014
(86) PCT No.: PCT/JP2014/061306

§ 371 Date: Oct. 22, 2015

(30) Foreign Application Priority Data
Apr. 30, 2013 (JP) 2013-095375

(51) Int. Cl. C07C 315/06; C07C 317/04

(52) U.S. Cl. CPC C07C 315/06; C07C 317/04

(57) ABSTRACT

A method of purifying dimethyl sulfoxide economically and at good yield satisfies the specifications of the United States Pharmacopoeia and reduces odor. The method includes distilling a solution having 35 parts by weight or more of water admixed per 100 parts by weight of raw dimethyl sulfoxide.

### TECHNICAL FIELD

This disclosure relates to a method of purifying dimethyl sulfoxide which is widely used as reaction solvent and synthesis reagent for medical and agrochemical intermediates and the like and, more specifically, relates to a method of economical, high-yield purification to provide high-quality dimethyl sulfoxide that is conformable to the U.S. Pharmacopeia and free of significant odors.

### BACKGROUND

Dimethyl sulfoxide has been widely used in industrial fields as a reaction solvent and synthesis reagent for medical and agrochemical intermediates, special detergents for electronic materials and the like. In particular, those dimethyl sulfoxide products specified in the U.S. Pharmacopeia are required to be higher in purity than the dimethyl sulfoxide products with quality for industrial use and also required to be free of significant odors.

Conventionally known methods of purifying dimethyl sulfoxide include a purification method in which dimethyl sulfoxide of general industry grade quality is brought into contact with activated carbon, followed by distillation treatment (see, for example, Japanese Examined Patent Publication (Kokoku) No. SHO 43-12128) and a purification method in which dimethyl sulfoxide of general industry grade quality is mixed with an organic solvent such as toluene to prepare a solution, followed by adding water, extracting dimethyl sulfoxide from the organic phase, and distillation (see, for example, U.S. Pat. No. 3,358,036).

However, the purification method proposed in Japanese Examined Patent Publication (Kokoku) No. SHO 43-12128 cannot remove odors. Although not specified in the U.S. Pharmacopeia, low odor emission is substantially an essential quality requirement for dimethyl sulfoxide products for medical use.

As dimethyl sulfoxide is highly soluble in both water and organic solvents, the purification method proposed in U.S. Pat. No. 3,358,036 is likely to suffer a large loss in the extraction step. Accordingly, a large quantity of water is necessary to reduce the loss and this large quantity of water has to be removed by distillation treatment, making the process uneconomical. Furthermore, although dimethyl sulfoxide products as specified in the Pharmacopoeia are administered directly to the human body, the purification method proposed in U.S. Pat. No. 3,358,036 uses organic solvents such as toluene that are harmful to the human body, leaving safety concerns about their use in drugs listed in the Pharmacopoeia.

It could therefore be helpful to provide a method of economical, high-yield purification to provide high-quality dimethyl sulfoxide conformable to the U.S. Pharmacopeia and free of significant odors.

### SUMMARY

We thus provide:

(1) A method of purifying dimethyl sulfoxide including a step for distilling a mixed solution containing 35 parts by weight or more of water and 100 parts by weight of dimethyl sulfoxide.

(2) A method of purifying dimethyl sulfoxide as described in paragraph (1) wherein the purified dimethyl sulfoxide has an absorbance of 0.01 to 0.20 at 275 nm.

(3) A method of purifying dimethyl sulfoxide as described in either paragraph (1) or (2) wherein the water is ion-exchanged water, distilled water, or ultrapure water.

(4) A method of purifying dimethyl sulfoxide as described in any one of paragraphs (1) to (3) wherein the water has an electrical conductivity of 50 mS/m or less at 25° C.

(5) A method of purifying dimethyl sulfoxide as described in any one of paragraphs (1) to (4) wherein the distillation of a mixed solution is carried out in a single step for evaporating water to obtain dimethyl sulfoxide.

(6) A method of purifying dimethyl sulfoxide as described in any one of paragraphs (1) to (4) wherein the distillation of a mixed solution is carried out in a first step for evaporating water to obtain roughly purified dimethyl sulfoxide, followed by a second step for distilling the roughly purified dimethyl sulfoxide to obtain purified dimethyl sulfoxide.

(7) High-purity dimethyl sulfoxide purified by a purification method as described in any one of paragraphs (1) to (6) and having a purity of 99.996% or more as determined by gas chromatograph (GC) analysis.

The dimethyl sulfoxide purification method improves absorbance, which represents a requirement specified in the U.S. Pharmacopeia, and largely decreases odor emissions, which represents a de facto quality requirement. Only water, which can be easily separated by distillation, is mixed with the dimethyl sulfoxide, making it possible to obtain a high-purity dimethyl sulfoxide with a high yield. In addition, the process is very economical because it does not require a large quantity of water.

### DETAILED DESCRIPTION

The dimethyl sulfoxide purification method meets the relevant requirements of the U.S. Pharmacopeia and provides economical, high-yield production of high-quality dimethyl sulfoxide free of significant odor emission. As stated above, when a conventional method is used to purify dimethyl sulfoxide of general industry grade, the purified dimethyl sulfoxide will still maintain unremoved odors and it is difficult to use in the medical context even if it meets the relevant requirements of the U.S. Pharmacopeia.

In this connection, the U.S. Pharmacopeia (U.S. Pharmacopeia USP36, NF31, p. 3269) specifies an absorbance at 275 nm as a quality requirement and European Pharmacopeia (European Pharmacopoeia 7.0) also specifies an absorbance at 275 nm. The difference in the absorbance at 275 nm represents the largest difference between general industry grade products and Pharmacopoeia-conformable ones and, accordingly, an absorbance at 275 nm as specified in the Pharmacopoeia is used herein as a primary quality item in quality evaluation of dimethyl sulfoxide. Determination of absorbance uses an ultraviolet absorptiometer, a cell with a width of a 1 cm, and water as a blank. A dimethyl sulfoxide specimen is first subjected to nitrogen bubbling for 30 minutes and measurement is performed within 10 minutes.

The odor of dimethyl sulfoxide is ranked according to the six-stage odor intensity criterion that has been used for odor evaluation in the Offensive Odor Control Act. This odor intensity ranking method ranks the odor intensity as 0 to 5 according to the six-stage odor intensity criterion shown below. The odor level of dimethyl sulfoxide can be evaluated according to this method.
Odor intensity 0: odorless
Odor intensity 1: barely perceivable odor
Odor intensity 2: identifiable but weak odor
Odor intensity 3: easily perceivable odor
Odor intensity 4: strong odor
Odor intensity 5: intense odor

To determine the odor of dimethyl sulfoxide, 15 mL of a dimethyl sulfoxide specimen was taken in a 30 mL sample bottle and the nose is brought close to the sample bottle at room temperature. Three or more testers sniff the odor from the same specimen and the rank agreed on by the largest number of testers was taken as the result of the odor intensity test.

There are no specific limitations on the raw materials, production method, and producer country of the dimethyl sulfoxide to be treated by the dimethyl sulfoxide purification method (hereinafter referred to as raw dimethyl sulfoxide). Preferably, it is dimethyl sulfoxide of general industry grade. There are no specific limitations on the purity of raw dimethyl sulfoxide, but it is preferably 99 wt % or more, more preferably 99.9 to 100 wt %. The use of raw dimethyl sulfoxide with such a purity provides high-quality dimethyl sulfoxide that is low in impurities content, conformable to the U.S. Pharmacopeia, and free of odors. The purity of dimethyl sulfoxide is measured with a gas chromatograph according to the method specified in the U.S. Pharmacopeia (U.S. Pharmacopeia USP36, NF31, p. 3269). Raw dimethyl sulfoxide may contain a compound other than dimethyl sulfoxide as long as it can be separated from the dimethyl sulfoxide by distillation.

The dimethyl sulfoxide purification method includes a step of distilling a mixed solution containing 35 parts by weight or more of water and 100 parts by weight of raw dimethyl sulfoxide. Mixing the raw dimethyl sulfoxide with water facilitates removal of impurities existing in trace amounts in the raw dimethyl sulfoxide and particularly reduces the odor intensity significantly. Reduction in the absorbance and odor of dimethyl sulfoxide cannot be easily realized if the content of water is not appropriate or if a solvent other than water is contained.

The water to be mixed with the raw dimethyl sulfoxide may be of any type generally available and it may be, for example, tap water, industrial water, pure water, steam-condensed water, distilled water, ion-exchanged water, or ultrapure water. The water is preferably conformable to A4 of JIS-K0557. Examples of water conformable to A4 of JIS-K0557 include distilled water, ion-exchanged water, and ultrapure water.

The water to be used preferably has an electrical conductivity of 50 mS/m or less, more preferably 30 mS/m or less, at 25° C. The use of water having such an electrical conductivity reduces the odor intensity of dimethyl sulfoxide. Generally, water conformable to A4 of JIS-K0557 has an electrical conductivity of 0.1 mS/m (25° C.). The electrical conductivity is measured at 25° C. according to JIS-K0130.

In regard to the addition of water to the raw dimethyl sulfoxide, the water should account for 35 parts by weight or more, preferably 50 parts by weight or more, relative to 100 parts by weight of the raw dimethyl sulfoxide. The odor reducing effect cannot be developed sufficiently if the content of water is smaller than 35 parts by weight. The upper limit of the water content is preferably 500 parts by weight, more preferably 300 parts by weight. If the quantity of water addition is more than 500 parts by weight, the purification step will be low in productivity, leading to industrial disadvantages.

Mixing raw dimethyl sulfoxide and water can be carried out by a commonly used method. The entire amount of water may be added to raw dimethyl sulfoxide at one time or divided into several parts. Alternatively, the entire amount of raw dimethyl sulfoxide may be added to water at one time or divided into several parts.

The distillation of a mixed solution of raw dimethyl sulfoxide and water may be carried out in one stage or in two stages to remove the water to obtain dimethyl sulfoxide. When the distillation is carried out in two stages, the first stage evaporates water from a mixed solution of raw dimethyl sulfoxide and water to provide a roughly purified dimethyl sulfoxide and the second stage distills the roughly purified dimethyl sulfoxide resulting from the first stage. Specifically, water is removed to provide roughly purified dimethyl sulfoxide in the first distillation stage and further purified dimethyl sulfoxide is obtained in the second distillation stage.

Distillation in the first stage may be performed by a commonly used distillation method such as, for example, high pressure distillation, low pressure distillation, reduced pressure distillation, molecular distillation, simple distillation, rectification, continuous distillation, and batch distillation, of which rectification under reduced pressure and simple distillation are preferable. Distillation in the second stage may be performed by high pressure distillation, low pressure distillation, reduced pressure distillation, molecular distillation, simple distillation, rectification, continuous distillation, batch distillation, or thin film distillation, of which rectification under reduced pressure, simple distillation, and thin film distillation are preferable.

Distillation of a mixed solution can be carried out in a single stage so that water is removed by evaporation to provide dimethyl sulfoxide. When the distillation is carried out in a single stage, the interior temperature is preferably 130° C. or less, more preferably 100° C. or less, and still more preferably 90° C. or less.

In a purification process in which the distillation is carried out in two stages, the first distillation stage removes water from a mixed solution by evaporation to provide roughly purified dimethyl sulfoxide. The subsequent second distillation stage distills the roughly purified dimethyl sulfoxide to provide purified dimethyl sulfoxide. For the first stage of this process, the interior temperature is preferably 130° C. or less, more preferably 100° C. or less, and still more preferably 90° C. or less. For the second stage, the interior temperature is preferably 130° C. or less, more preferably 110° C. or less.

Reduced pressure distillation is preferably performed at a pressure 14.2 kPa or less, more preferably 6.7 kPa or less. If the distillation temperature or pressure is high, decomposition of dimethyl sulfoxide will be accelerated, possibly making it impossible to depress the aggravation of odors.

The purification method described above can provide high-quality dimethyl sulfoxide conformable to the Pharmacopoeia and free of significant odors and has a very high chemical purity. It is preferable for this high purity dimethyl sulfoxide to have a purity of preferably 99.996% or more as determined by gas chromatograph (GC) analysis.

### EXAMPLES

Our methods will now be illustrated in more detail with reference to examples, but it should be understood that this disclosure is not construed as being limited to these examples.

For these examples, the chemical purity of raw dimethyl sulfoxide and the absorbance and odor intensity of dimethyl sulfoxide resulting from purification treatment (hereinafter referred to as purified dimethyl sulfoxide) and raw dimethyl sulfoxide were measured as described above.

### Method of Measuring the Chemical Purity of Raw Dimethyl Sulfoxide

The chemical purity of raw dimethyl sulfoxide was measured using a gas chromatograph (GC-2010, manufactured by Shimadzu) under the following conditions.
Analysis equipment: GC (GC-2010, manufactured by Shimadzu)
Carrier gas: helium (column flow rate 1.7 mL/min)
Analysis column: Rxt-1 (15 m×0.32 mm×3 μm) (manufactured by RESTEK)
Column temperature: 100° C. (15 min)-->(10° C./min)-->170° C. (20 min)
Inlet temperature: 210° C.
Injection volume: 1 μL
Detector: FID
Detector temperature: 220° C.

### Method of Measuring Impurities in Purified Dimethyl Sulfoxide

Mass analysis was performed using a gas chromatograph (7890A, manufactured by Agilent) under the following conditions and unidentifiable impurities detected at a retention time of 4.3 minutes were examined.
Analysis equipment: GC (7890A, manufactured by Agilent)
Carrier gas: helium (column flow rate 2.0 mL/min)
Analysis column: Stabilwax (30 m×0.32 mm×0.5 μm) (manufactured by RESTEK)
Column temperature: 35° C. (3 min)-->(7° C./min)-->130° C. (10 min)-->(7° C./min)-->160° C.-->(15° C./min)-->250° C. (8 min)
Inlet temperature: 200° C.
Injection volume: 1 μL
Detector: FID
Detector temperature: 270° C.

### Method of Measuring Absorbance of Purified Dimethyl Sulfoxide and Raw Dimethyl Sulfoxide

Dimethyl sulfoxide was first subjected to nitrogen bubbling for 30 minutes and within 10 minutes, the absorbance at 275 nm was measured using an ultraviolet absorptiometer (UV-1800, manufactured by Shimadzu) under the following conditions.
Analysis equipment: ultraviolet absorptiometer
Cell: quartz, width 1 cm
Blank: water

### Method of Measuring Odor Intensity of Purified Dimethyl Sulfoxide and Raw Dimethyl Sulfoxide

Sensory evaluation of the odor of dimethyl sulfoxide was performed by three testers according to the following six-stage odor intensity criterion. In a 30 mL sample bottle, 15 mL of dimethyl sulfoxide was taken to provide a specimen. Each of the three testers brought their noses close to the sample bottle at room temperature, sniffed the odor from the same specimen, and ranked the odor intensity according to the following six-stage (rank 0 to 5) criterion. The rank agreed on by the largest number of testers was taken as the odor intensity of the specimen.
Odor intensity 0: odorless
Odor intensity 1: barely perceivable odor
Odor intensity 2: identifiable but weak odor
Odor intensity 3: easily perceivable odor
Odor intensity 4: strong odor
Odor intensity 5: intense odor

### Example 1

In a 200 mL round bottom flask, 50 g of dimethyl sulfoxide of general industry grade with a purity of 99.9 wt % (absorbance 0.22 at 275 nm and odor intensity 3) and 50 g (100 parts by weight relative to 100 parts by weight of dimethyl sulfoxide) of ion-exchanged water conformable to A4 of JIS-K0557 were fed and mixed. A rectifying tube with a diameter of 3 cm and a height of 15 cm containing Raschig rings (each having a length of 5 mm, outside diameter of 5 mm, and inside diameter of 3 mm) installed over a total length of 10 cm was fitted on the mouth of the round bottom flask and distillation was performed at a reduced pressure of 6.7 kPa. Heating the round bottom flask was started at an internal temperature of 45° C. and the receiver was replaced when a temperature arrived to 107° C., thus providing 40 g of purified dimethyl sulfoxide. At this point, the distillation yield was 80%. The resulting purified dimethyl sulfoxide exhibited an absorbance at 275 nm of 0.12 and an odor intensity of 1. These results are summarized in Table 1. In the table, the quantity of water added is shown in terms of parts by weight relative to 100 parts by weight of dimethyl sulfoxide.

### Examples 2 to 5

Except for adding water in the amount shown in Table 1, the same purification procedure as in Example 1 was carried out. The distillation yield was 81%, 80%, 70%, and 79% in Examples 2, 3, 4, and 5, respectively. Qualities of the resulting purified dimethyl sulfoxide samples are described in Table 1.

### Examples 6 and 7

Except for using distilled water or ultrapure water conformable to A4 of JIS-K0557 instead of the water added in Example 1, the same purification procedure as in Example 1 was carried out. The distillation yield was 79% and 81% in Examples 6 and 7, respectively. Qualities of the resulting purified dimethyl sulfoxide samples are described in Table 1.

TABLE 1

| | | | Example 1 | Example 2 | Example 3 | Example 4 | Example 5 | Example 6 | Example 7 |
|---|---|---|---|---|---|---|---|---|---|
| Purity of raw dimethyl | | wt % | 99.9 | 99.9 | 99.9 | 99.9 | 99.9 | 99.9 | 99.9 |
| sulfoxide | | | | | | | | | |
| Type of water | | (—) | ion-exchanged | ion-exchanged | ion-exchanged | ion-exchanged | ion-exchanged | distilled | ultrapure |
| | | | water | water | water | water | water | water | water |
| Amount of water added | | parts by | 100 | 50 | 200 | 40 | 35 | 100 | 100 |
| | | weight | | | | | | | |
| Internal temperature during | | ° C. | 45-107 | 45-107 | 45-107 | 45-107 | 45-107 | 45-107 | 45-107 |
| distillation | | | | | | | | | |
| Quality | Absorbance | (—) | 0.12 | 0.13 | 0.08 | 0.14 | 0.14 | 0.12 | 0.12 |
| | Odor intensity | (—) | 1 | 1 | 1 | 2 | 2 | 1 | 1 |

Note)
The ion-exchanged water, distilled water, and ultrapure water included here are all conformable to A4 of JIS K0557.

### Comparative Examples 1 to 4

In Comparative examples 1, 2, and 4, the same procedure as in Example 1 was carried out except for adding water in the amount shown in Table 2. In Comparative example 3, the same procedure as in Example 1 was carried out except for adding methanol (guaranteed reagent, manufactured by Nacalai Tesque, Inc.) instead of water. The distillation yield was 85%, 95%, 95%, and 79% in Comparative examples 1, 2, 3, and 4, respectively. Qualities of the resulting dimethyl sulfoxide samples are described in Table 2.

TABLE 2

| | | | Comparative | Comparative | Comparative | Comparative |
|---|---|---|---|---|---|---|
| | | | example 1 | example 2 | example 3 | example 4 |
| Purity of raw dimethyl | | wt % | 99.9 | 99.9 | 99.9 | 99.9 |
| sulfoxide | | | | | | |
| Type of liquid added | | (—) | ion-exchanged | none | methanol | ion-exchanged |
| | | | water | | | water |
| Amount of liquid added | | parts by | 10 | 0 | 100 | 33 |
| | | weight | | | | |
| Internal temperature during | | ° C. | 45-107 | 45-107 | 45-107 | 45-107 |
| distillation | | | | | | |
| Quality | Absorbance | (—) | 0.16 | 0.22 | 0.21 | 0.14 |
| | Odor intensity | (—) | 3 | 3 | 3 | 3 |

Note)
The ion-exchanged water included here is conformable to A4 of JIS K0557.

### Example 8

In a 200 mL round bottom flask, 50 g of dimethyl sulfoxide of general industry grade with a purity of 99.9 wt % (absorbance 0.22 at 275 nm and odor intensity 3) and 50 g (100 parts by weight relative to 100 parts by weight of dimethyl sulfoxide) of tap water were fed and mixed. The tap water used had an electrical conductivity of 13 mS/m (25° C.). A rectifying tube with a diameter of 3 cm and a height of 15 cm containing Raschig rings (each having a length of 5 mm, outside diameter of 5 mm, and inside diameter of 3 mm) installed over a total length of 10 cm was fitted on the mouth of the round bottom flask and distillation was performed at a reduced pressure of 6.7 kPa. The round bottom flask was heated from an internal temperature of 45° C. to 80° C. and the distillation was stopped at the time when the water content in the distillate came to 0.1% or below. The dimethyl sulfoxide left after removing water exhibited an absorbance at 275 nm of 0.11 and an odor intensity of 1. These results are summarized in Table 3. In the table, the quantity of water added is shown in terms of parts by weight relative to 100 parts by weight of dimethyl sulfoxide.

### Examples 9 and 10

Except for adding water in the amount shown in Table 3, the same distillation procedure as in Example 8 was carried out. Qualities of the resulting dimethyl sulfoxide samples are described in Table 3.

### Examples 11 and 12

After heating the round bottom flask from an internal temperature of 45° C. to 70° C., simple distillation was performed instead of using a rectifying tube as in Example 8 and water was added in the amount described in Table 3. Except for these changes, the same distillation procedure as in Example 8 was carried out. Qualities of the resulting dimethyl sulfoxide samples are described in Table 3.

TABLE 3

| | | | Example 8 | Example 9 | Example 10 | Example 11 | Example 12 |
|---|---|---|---|---|---|---|---|
| Purity of raw dimethyl | | wt % | 99.9 | 99.9 | 99.9 | 99.9 | 99.9 |
| sulfoxide | | | | | | | |
| Type of water | | (—) | tap water | tap water | tap water | tap water | tap water |
| Amount of water added | | parts by | 100 | 150 | 200 | 150 | 200 |
| | | weight | | | | | |
| Distillation conditions | | (—) | rectification | rectification | rectification | simple | simple |
| | | | | | | distillation | distillation |
| Internal temperature during | | ° C. | 45-80 | 45-80 | 45-80 | 45-70 | 45-70 |
| distillation | | | | | | | |
| Quality | Absorbance | (—) | 0.11 | 0.10 | 0.12 | 0.10 | 0.12 |
| | Odor intensity | (—) | 1 | 1 | 1 | 1 | 1 |

### Example 13

In a 200 mL round bottom flask, 50 g of dimethyl sulfoxide of general industry grade with a purity of 99.9 wt % (absorbance 0.22 at 275 nm and odor intensity 3) and 50 g (100 parts by weight relative to 100 parts by weight of dimethyl sulfoxide) of tap water were fed to prepare a mixed solution. A rectifying tube with a diameter of 3 cm and a height of 15 cm containing Raschig rings (each having a length of 5 mm, outside diameter of 5 mm, and inside diameter of 3 mm) installed over a total length of 10 cm was fitted on the mouth of the round bottom flask and distillation was performed at a reduced pressure of 6.7 kPa as the first-stage distillation for the mixed solution. The round bottom flask was heated from an internal temperature of 45° C. to 80° C. and the distillation was stopped at the time when the water content in the distillate came to 0.1% or below. The roughly purified dimethyl sulfoxide left after removing water exhibited an absorbance at 275 nm of 0.11 and an odor intensity of 1.

For the second-stage distillation, the roughly purified dimethyl sulfoxide obtained from the first-stage distillation was fed in a 200 mL round bottom flask and a rectifying tube with a diameter of 3 cm and a height of 15 cm containing Raschig rings (each having a length of 5 mm, outside diameter of 5 mm, and inside diameter of 3 mm) installed over a total length of 10 cm was fitted, followed by heating the round bottom flask from an internal temperature of 45° C. to 107° C. and distillation at a reduced pressure of 6.7 kPa. The purified dimethyl sulfoxide obtained from the second-stage distillation exhibited an absorbance at 275 nm of 0.09 and an odor intensity of 1. These results are summarized in Table 4.

### Example 14

Except for adding water in the amount shown in Table 4 instead of the amount adopted in Example 13, the same first- and second-stage distillation procedures as in Example 13 were carried out. The roughly purified dimethyl sulfoxide obtained from the first-stage distillation exhibited an absorbance at 275 nm of 0.10, an odor intensity of 1, and a chemical purity of 99.9957% as determined by GC analysis. The purified dimethyl sulfoxide obtained from the second-stage distillation exhibited an absorbance at 275 nm of 0.08, an odor intensity of 1, and a chemical purity of 99.9989% as determined by GC analysis. These results are summarized in Table 4.

### Example 15

Except for adding water in the amount shown in Table 4 instead of the amount adopted in Example 13, the same first- and second-stage distillation procedures as in Example 13 were carried out. The roughly purified dimethyl sulfoxide obtained from the first-stage distillation exhibited an absorbance at 275 nm of 0.12, an odor intensity of 1, and a chemical purity of 99.9957% as determined by GC analysis. The purified dimethyl sulfoxide obtained from the second-stage distillation exhibited an absorbance at 275 nm of 0.08, an odor intensity of 1, and a chemical purity of 99.9980% as determined by GC analysis. These results are summarized in Table 4.

### Example 16

Water was added in the amount shown in Table 4 instead of the amount adopted in Example 13; no rectifying tube was used in the first-stage distillation procedure, in which simple distillation was performed after heating the round bottom flask from an internal temperature of 45° C. to 70° C.; and no rectifying tube was used in the second-stage distillation procedure, in which simple distillation was performed after heating the round bottom flask from an internal temperature of 45° C. to 80° C. Except for these changes, the same distillation procedures as in Example 13 were carried out. Qualities of the resulting purified dimethyl sulfoxide sample are described in Table 4.

### Example 17

Except for adding water in the amount shown in Table 4 instead of the amount adopted in Example 16, the same first- and second-stage distillation procedures as in Example 16 were carried out. Qualities of the resulting purified dimethyl sulfoxide sample are described in Table 4.

### Example 18

Water was added in the amount shown in Table 4 instead of the amount adopted in Example 13, and no rectifying tube was used in the second-stage distillation procedure in which thin film distillation was performed after heating the round bottom flask from an internal temperature of 80° C. to 110° C. Except for these changes, the same distillation procedures as in Example 13 were carried out. Qualities of the resulting purified dimethyl sulfoxide sample are described in Table 4.

TABLE 4

| | | | | Example 13 | Example 14 | Example 15 | Example 16 | Example 17 | Example 18 |
|---|---|---|---|---|---|---|---|---|---|
| Distillation | Purity of raw dimethyl | | wt % | 99.9 | 99.9 | 99.9 | 99.9 | 99.9 | 99.9 |
| conditions | sulfoxide | | | | | | | | |
| (first stage) | Type of water | | (—) | tap water | tap water | tap water | tap water | tap water | tap water |
| | Amount of water added | | parts by | 100 | 150 | 200 | 150 | 200 | 200 |
| | | | weight | | | | | | |
| | Distillation conditions | | (—) | rectification | rectification | rectification | simple | simple | rectification |
| | | | | | | | distillation | distillation | |
| | Internal temperature during | | ° C. | 45-80 | 45-80 | 45-80 | 45-70 | 45-70 | 45-80 |
| | distillation | | | | | | | | |
| | Quality | Absorbance | (—) | 0.11 | 0.10 | 0.12 | 0.10 | 0.12 | 0.12 |
| | | Odor intensity | (—) | 1 | 1 | 1 | 1 | 1 | 1 |
| Distillation | Distillation conditions | | (—) | rectification | rectification | rectification | simple | simple | thin film |
| conditions | | | | | | | distillation | distillation | distillation |
| (second stage) | Internal temperature during | | ° C. | 45-107 | 45-107 | 45-107 | 45-80 | 45-80 | 80-110 |
| | distillation | | | | | | | | |
| | Quality | Absorbance | (—) | 0.09 | 0.08 | 0.08 | 0.08 | 0.08 | 0.10 |
| | | Odor intensity | (—) | 1 | 1 | 1 | 1 | 1 | 1 |

1.-7. (canceled)

8. A method of purifying dimethyl sulfoxide comprising distilling a mixed solution prepared by adding 35 parts by weight or more of water to 100 parts by weight of raw dimethyl sulfoxide.

9. The method as described in claim 8, wherein the purified dimethyl sulfoxide has an absorbance of 0.01 to 0.20 at 275 nm.

10. The method as described in claim 8, wherein the water is ion-exchanged water, distilled water, ultrapure water, or tap water.

11. The method as described in claim 8, wherein the water has an electrical conductivity of 50 mS/m or less at 25° C.

12. The method as described in claim 8, wherein the distillation is carried out in a single step to evaporate water to obtain dimethyl sulfoxide.

13. The method as described in claim 8, wherein the distillation is carried out in a first step to evaporate water to obtain roughly purified dimethyl sulfoxide, followed by a second step of distilling the roughly purified dimethyl sulfoxide to obtain purified dimethyl sulfoxide.

14. High-purity dimethyl sulfoxide obtained by a purification method as described in claim 8 and having a purity of 99.996% or more as determined by gas chromatograph (GC) analysis.

15. The method as described in claim 9, wherein the water is ion-exchanged water, distilled water, ultrapure water, or tap water.

16. The method as described in claim 9, wherein the water has an electrical conductivity of 50 mS/m or less at 25° C.

17. The method as described in claim 10, wherein the water has an electrical conductivity of 50 mS/m or less at 25° C.

18. The method as described in claim 9, wherein the distillation is carried out in a single step to evaporate water to obtain dimethyl sulfoxide.

19. The method as described in claim 10, wherein the distillation is carried out in a single step to evaporate water to obtain dimethyl sulfoxide.

20. The method as described in claim 11, wherein the distillation is carried out in a single step to evaporate water to obtain dimethyl sulfoxide.

21. The method as described in claim 9, wherein the distillation is carried out in a first step to evaporate water to obtain roughly purified dimethyl sulfoxide, followed by a second step of distilling the roughly purified dimethyl sulfoxide to obtain purified dimethyl sulfoxide.

22. The method as described in claim 10, wherein the distillation is carried out in a first step to evaporate water to obtain roughly purified dimethyl sulfoxide, followed by a second step of distilling the roughly purified dimethyl sulfoxide to obtain purified dimethyl sulfoxide.

23. The method as described in claim 11, wherein the distillation is carried out in a first step to evaporate water to obtain roughly purified dimethyl sulfoxide, followed by a second step of distilling the roughly purified dimethyl sulfoxide to obtain purified dimethyl sulfoxide.

24. High-purity dimethyl sulfoxide obtained by a purification method as described in claim 9 and having a purity of 99.996% or more as determined by gas chromatograph (GC) analysis.

25. High-purity dimethyl sulfoxide obtained by a purification method as described in claim 10 and having a purity of 99.996% or more as determined by gas chromatograph (GC) analysis.

26. High-purity dimethyl sulfoxide obtained by a purification method as described in claim 11 and having a purity of 99.996% or more as determined by gas chromatograph (GC) analysis.

27. High-purity dimethyl sulfoxide obtained by a purification method as described in claim 12 and having a purity of 99.996% or more as determined by gas chromatograph (GC) analysis.
